(19) United States
(12) Patent Application Publication
(10) Pub. No.: US 2025/0114630 A1
(43) Pub. Date: Apr. 10, 2025

(54) Multi-Wavelength LED Phototherapy Device with Integrated Mobile App Control

(71) Applicant: Aharon Zaide, Dubai (AE)

(72) Inventor: Aharon Zaide, Dubai (AE)

(21) Appl. No.: 18/376,916

(22) Filed: Oct. 5, 2023

(51) Int. Cl. A61N 5/06

(52) U.S. Cl. CPC A61N 5/0616; A61N 2005/0626; A61N 2005/0652; A61N 2005/0653; A61N 2005/0663

(57) ABSTRACT

The invention describes a Multi-Wavelength LED Phototherapy Device with Integrated Mobile App Control. This device consists of a silicone body embedded with LED beads, placed atop a base and operated via a remote control. The silicone body showcases a transparent silicon logo and features layers including a first layer, a secondary translucent silicon layer, and an RGBW LED light array with 888 LED beads. Additional features consist of an OLED light matching color 8262C and a 5-pin female port. The base incorporates various connectors, an IR remote receiver, a lighted power button, LED edge lighting, and a battery. Connectivity options for WiFi and IR remote applications are present, along with anti-slip rubber at the bottom. The invention's application extends to therapeutic treatments using diverse wavelengths across the visible spectrum

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### Field of the Invention

The present invention relates generally to the field of medical devices and more specifically to skin care and treatment. Even more particularly, the invention relates to phototherapy devices that utilize light-emitting diodes (LEDs) to emit multiple wavelengths of light for the treatment of various skin conditions. The invention incorporates an integrated mobile application and remote control for personalized treatment options.

### Description of the Related Art

The World Medical Association (WMA) has provided corroborative findings that specific wavelengths of light have the ability to enhance the vitality and general health of skin cells and tissues. Over the years, this has given rise to the development of phototherapy devices, which are medical-grade devices designed to utilize light-emitting diodes (LEDs) in a targeted manner to treat a plethora of skin conditions, such as acne, sunburn, psoriasis, and signs of aging like wrinkles and fine lines.

Traditional phototherapy devices have leveraged biophototherapy principles with nascent nanotechnology to guide light materials onto the skin surface. These devices generally do not produce excessive heat, making them tolerable for users. They leverage low-energy pure light and are often equipped with tactile light adjustment technology for skin care. Treatments are marketed as natural, mild, and low-risk, making these devices versatile across all skin types. The mode of action involves the absorption of light by skin cells, where it gets converted into intracellular energy. This leads to various biochemical processes, such as the enzymatic reactions that increase cellular “guards” like catalase and superoxide dismutase (SOD), and the breakdown of Adenosine Triphosphate (ATP), the energy currency of the cell, thereby promoting cell metabolism and synthesis.

Despite these promising attributes, there are limitations in existing phototherapy devices that warrant attention and provide the impetus for the current invention.

The traditional devices predominantly use a single type of light source, usually one form of LED to produce a monochromatic light of a fixed wavelength. Such single-wavelength devices have proven effective for treating specific conditions but suffer from an inherent lack of versatility. For example, a device equipped only with a blue LED light (approx. 470 nm) would be effective against acne but not necessarily for treating wrinkles or increasing skin elasticity.

To add versatility, some advanced phototherapy systems have employed multiple types of LEDs or used optical filters to alter the emitted wavelengths. While this solves the issue of versatility to some extent, these multi-wavelength devices are usually complex, difficult to maintain, and expensive. The optical filters or mechanical parts used for switching light sources can wear out over time, requiring costly replacements and regular maintenance.

Furthermore, existing phototherapy devices are generally bulky and are not designed with the end-user's convenience in mind. Most lack sophisticated control mechanisms and offer limited customization options, often needing manual adjustments during treatment. This lack of user-friendly interfaces extends to the absence of mobile application-based or remote control features, limiting user interaction and customization options.

In terms of safety and comfort, although many current devices are designed to minimize heat generation, there still exists a category that utilizes high-intensity light sources. These can produce excessive heat, making treatments uncomfortable and potentially risky, particularly for sensitive skin types or prolonged exposure. Thus, while existing devices have laid a significant foundation in the realm of phototherapy, there is still considerable room for innovation. Specifically, there is a need for a device that combines the versatility of multiple light wavelengths, the convenience of user-friendly controls, and the assurance of safety for all skin types.

### SUMMARY OF THE INVENTION

The present invention addresses the limitations inherent in existing phototherapy devices by introducing a highly versatile, user-friendly, and efficient system for treating a multitude of skin conditions. The Multi-Wavelength LED Phototherapy Device with Integrated Mobile App Control of the present invention is uniquely equipped with 888 LED beads, each carefully engineered to emit light at multiple wavelengths, thereby offering a broader range of treatment options within a single, integrated device.

The invention distinguishes itself by featuring seven distinct light sources: red, blue, green, yellow, purple, cyan, and laser (white) light, each providing specialized therapeutic effects. This multifaceted approach allows for targeted treatment of various skin conditions such as acne, inflammation, wrinkles, and pigmentation, among others. Users can easily switch between different light sources depending on their specific needs, eliminating the need for multiple devices or complex modifications.

Moreover, the present invention is designed for ease of use and convenience. It is equipped with an integrated mobile application and a remote control system, allowing users to customize their treatment parameters with ease. This adds a level of personalization and convenience missing in existing phototherapy devices, which often require manual adjustments and lack user-friendly interfaces.

Another key innovation is the device's intelligent heat management system. Unlike conventional high-intensity light sources that generate excessive heat, the current invention utilizes advanced thermal management techniques to ensure that the emitted light remains at low, safe temperatures, thereby reducing the risk of skin damage and enhancing user comfort.

Furthermore, the construction of the present invention includes a silicone body designed for enhanced durability and user comfort, an OLED light display, a 5-pin female port for additional accessories, and a base with various functionalities including an IR remote receiver and WiFi capabilities for enhanced connectivity options.

In summary, the present invention offers a comprehensive solution to the limitations of existing phototherapy technology by delivering a versatile, user-friendly, and efficient system capable of treating a broad array of skin conditions. Through its advanced features and thoughtful design, it sets a new standard in the realm of phototherapy devices

### DESCRIPTION OF THE DRAWINGS

The accompanying drawings, which are included to provide further understanding and are incorporated in and constitute a part of this specification, illustrate embodiments of the invention and, together with the description, serve to explain the principles of the invention.

FIG. 1 This is a top perspective view of the present invention, showcasing the silicone body housing the LED beads, the base that contains various electronic components, and the remote control used for operating the device.

FIG. 2 This is an exploded view detailing the silicone body and LED beads. The drawing illustrates the transparent silicon logo, the white silicon (RAL 9003), and the arrangement of the LED beads.

FIG. 3 This figure provides another exploded view of the silicone body and LED beads, emphasizing different components. It shows the translucent silicon, RGBW LED light array with a total of 888 LED beads, OLED light to match color 8262C, slot for OLED, and a 5-pin female port for additional features or future expandability.

FIG. 4 An exploded view of the base is provided in this figure. The base contains various components including the cable to a 5-pin connector (for RGBW LED) for the silicon pad, cable to IR remote receiver, IR remote receiver, aluminum electroplated finish color (PANTONE 8262C), lighted power button, LED edge light, cable from charging port, battery, and electronic component for WiFi and IR remote application. The base is finished in matte white aluminum (RAL 9003).

FIG. 5 This figure shows another exploded view of the base, focusing on the 5-pin power input, the 5-pin connector (for RGBW LED lights), and the charging/power port.

FIG. 6 A bottom perspective view of the base is shown in this figure, focusing on features like the anti-slip rubber and placement of screws (manufacturer to coordinate suitable location).

FIG. 7 This figure provides a top perspective view of the base, showcasing its ergonomic design and user-friendly interface.

FIG. 8 This is a top view of the remote control, detailing its construction. The remote is made from white silicon (RAL 9003) and ABS with electroplating finish color (PANTONE 8262C). It features a time display programmed to a maximum of 30 minutes, a color display programmed to the seven colors, silicon buttons (white RAL 9003), and a light grey printed power symbol.

The figures are designed to offer a comprehensive visual understanding of the present invention, thereby complementing the detailed description of the invention provided herein.

### REFERENCE NUMBERS OF THE DRAWINGS

The following is a list of reference numbers used in the drawings to identify various components and elements of the present invention. These numbers serve to correlate the elements across multiple figures for a unified understanding.

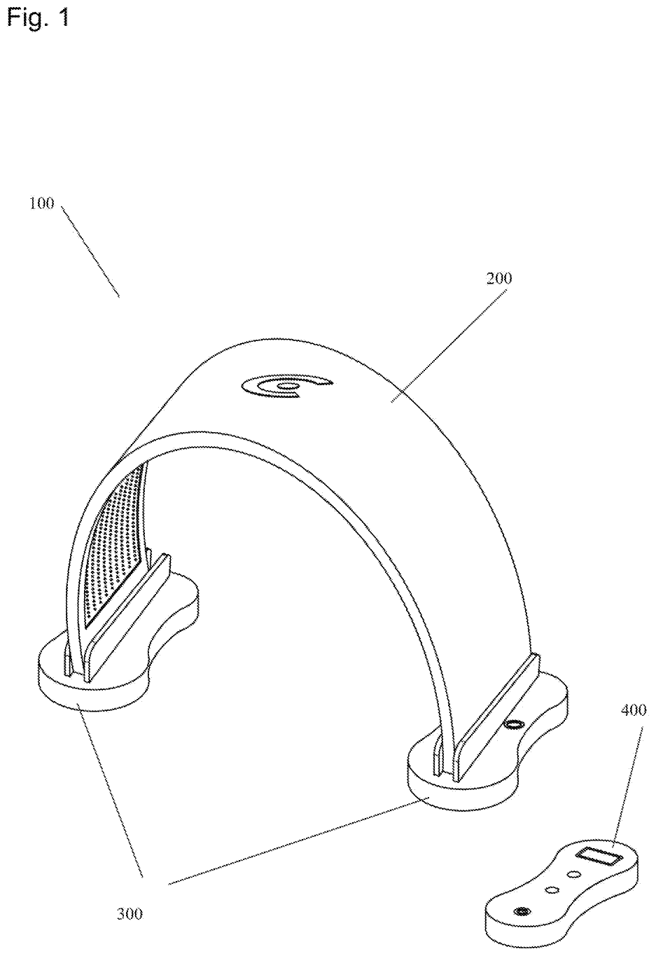

### FIG. 1—Top Perspective View of the Present Invention

100: Multi-Wavelength LED Phototherapy Device with Integrated Mobile App Control
200: Silicone body and LEDs beads
300: Base
400: Remote control

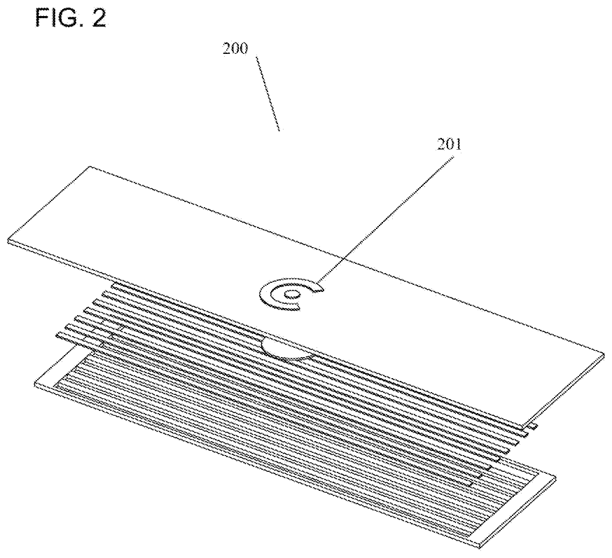

### FIG. 2—Exploded View of Silicone Body and LEDs Beads

201: Transparent Silicon Logo

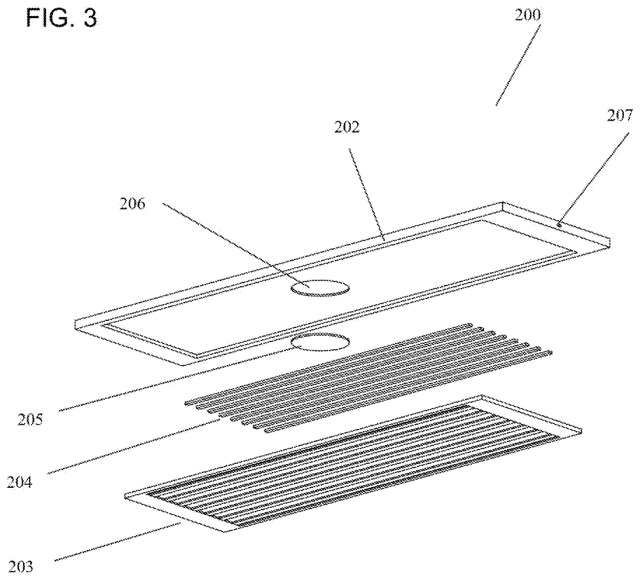

### FIG. 3—Exploded View of Silicone Body and LEDs Beads

202: First layer
203: A secondary layer of translucent silicon
204: RGBW LED Light Array with Total 888 LED beads
205: OLED Light to Match Color 8262C
206: Slot for OLED
207: 5-Pin Female Port

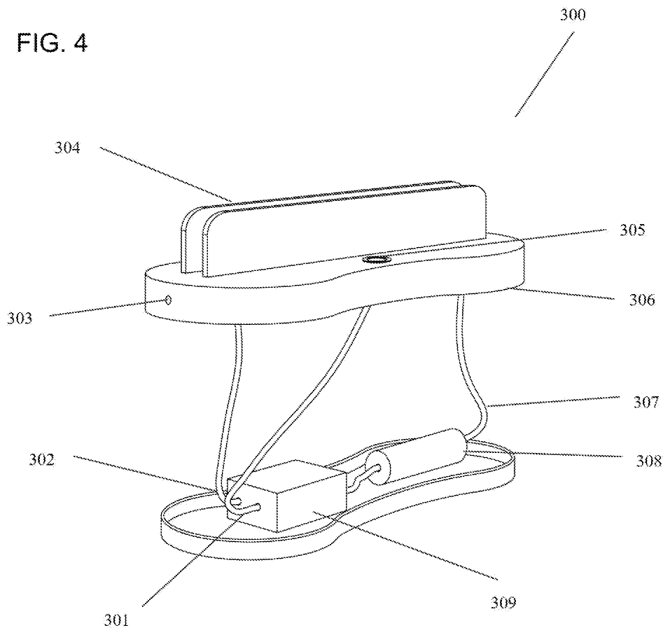

### FIG. 4—Exploded View of the Base

301: Cable to 5-Pin Connector (For RGBW LED) for Silicon Pad
302: Cable to IR Remote Receiver
303: IR Remote Receiver
305: Lighted Power Button
306: LED Edge Light
307: Cable from Charging Port
308: Battery
309: Electronic Component for WiFi and IR Remote Application

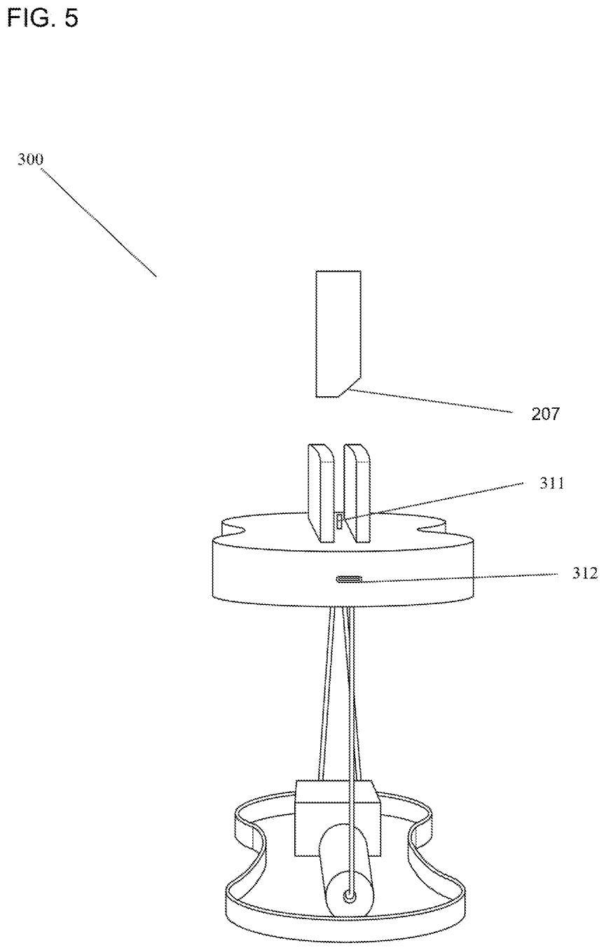

### FIG. 5—Exploded View of the Base

311: 5-Pin Connector (For RGBW LED Lights)
312: Charging/Power Port

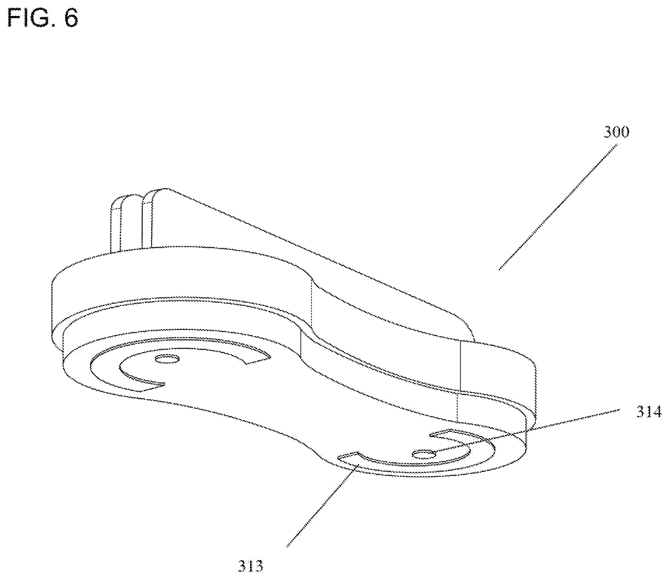

### FIG. 6—Bottom Perspective View of the Base

313: Anti-Slip Rubber
314: Screw Location (Manufacturer to Coordinate Suitable Location)

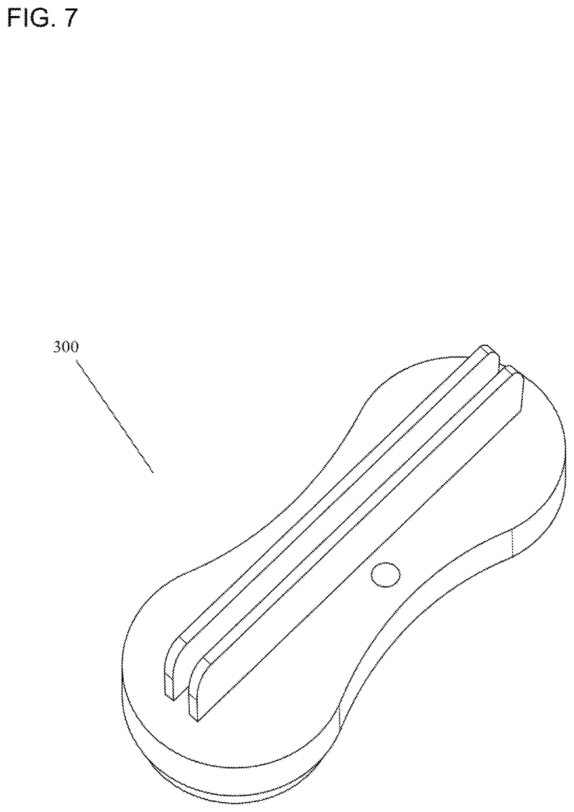

### FIG. 7—Top Perspective View of the Base

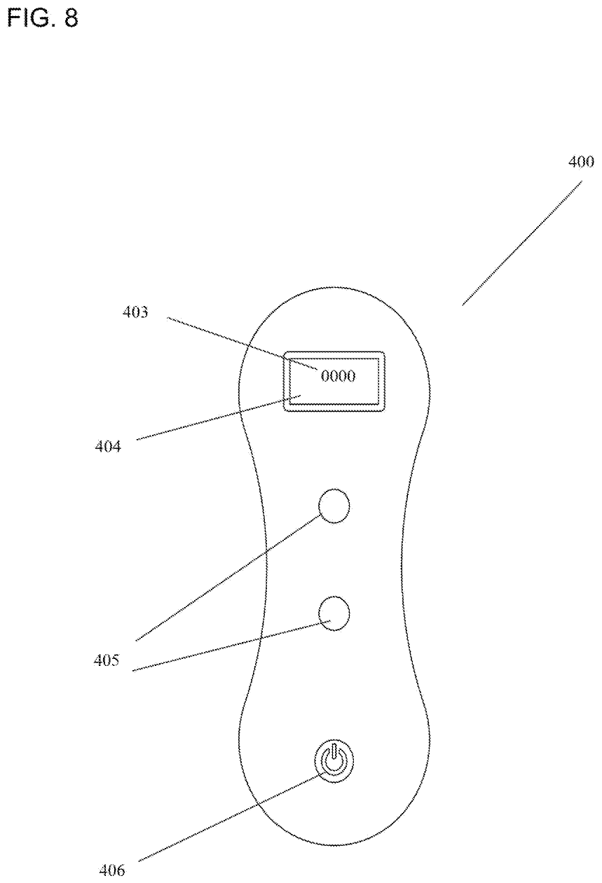

### FIG. 8—Top View of the Remote Control

403: Time Display
404: Color Display
405: Silicon Buttons (White RAL 9003)
406: Light Grey Printed Power Symbol

By referring to these numbers, one can easily correlate the components across different drawings for a cohesive understanding of the device's construction and functionality.

### DETAILED DESCRIPTION OF THE INVENTION

In the following detailed description, reference is made to the accompanying drawings that form a part thereof, and which is shown by way of illustration specific embodiments in which the invention may be practiced. These embodiments are described in sufficient detail to enable those skilled in the art to practice the invention, and it is to be understood that other embodiments may be utilized and that structural, logical, and electrical changes may be made without departing from the scope of the present invention.

### Overview

Referring to FIG. 1 the present invention, designated as a multi-wavelength LED phototherapy device with integrated mobile app control 100, aims to offer an advanced skin treatment solution using phototherapy technology. The device utilizes a total of 888 LED beads that emit multiple wavelengths of light for various skincare treatments.

Silicone body and LEDs beads 200, 201, 202, 203, 204, 205, 206 and 207. Referring to FIG. 2 and FIG. 3, the silicone body 200 serves as the housing for the LED beads 204. The Transparent Silicon Logo 201 is aesthetically placed on the surface, offering brand visibility. The silicone body 200 is made from White Silicon, which provides a pleasing aesthetic as well as ensures skin-safety.

A secondary layer of translucent silicon 203 is also present, aiding in the diffusion of the LED lights. The device utilizes an RGBW LED light array containing a total of 888 LED beads 204, strategically arranged to offer maximum skin exposure for effective treatment. The LED array is complemented by an OLED Light 205 that matches the Color 8262C. A Slot for OLED 206 is incorporated in the silicone body to accommodate the OLED component. Additionally, a 5-pin female port 207 is situated within the first layer 202, which serves as the connection interface for power and data transmission.

The base of the device 300, 301, 302, 303, 304, 305, 306, 307, 308, 309, 310, 311, 312, 313 and 314.

As illustrated in FIG. 4, FIG. 5, and FIG. 6, the base 300 includes several features. A cable to a 5-Pin connector 301 is used to link the LED light array 204 to the base 300. Additionally, another cable to IR remote receiver 302 is present for communication with the remote control 400. The base 300 includes an IR remote receiver 303 that is designed to pick up signals from the remote control 400. The base 300 has an aluminum electroplated finish in color PANTONE 8262C, which not only provides aesthetic value but also ensures durability. It features a Lighted power button 305 that allows users to power the Multi-Wavelength LED phototherapy device with integrated mobile app control 100 on or off manually. LED edge lighting 306 is included for visual appeal and functional indicators. The cable from the charging port 307 facilitates the power supply to the Multi-Wavelength LED phototherapy device with Integrated mobile app control 100.

An integrated Battery 308 provides the power reserve, and an Electronic Component for WiFi and IR Remote Application 309 controls the wireless functionalities. The base 300 has a 5-Pin Connector for RGBW LED Lights 311 that connects to the 5-Pin Female Port 207 of the silicone body and LEDs beads 200. Finally, a charging/power Port 312 is included for electrical connection, and anti-slip rubber 313 and a screw location 314 ensure stable placement.

Remote control 400, 403, 404, 405 and 406.

As depicted in FIG. 8, the remote control 400 is designed for ease of use, featuring a casing made of white silicon and ABS with an electroplating finish in color PANTONE 8262C. The Remote control 400 offers a time display 403 programmed to a maximum of 30 minutes and a color display 404 programmed for 7 colors. The device features silicon buttons 405, white RAL 9003 for user input, and a light grey printed power symbol 406 for ease of identification.

### Integrated Mobile Application for Device Control

One of the groundbreaking features of the multi-wavelength LED phototherapy device 100 is its seamless integration with a bespoke mobile application. This mobile application is designed to offer users an enhanced level of control and customization that is not typically available in traditional phototherapy devices.

### Application Features and User Interface:

The mobile application presents a clean, intuitive user interface that allows users to toggle between different wavelengths of light emitted by the RGBW LED Light Array with Total 888 LED beads 204 in the silicone body 200.

Users can pre-program treatment sessions, specifying the wavelength, intensity, and duration through the app. Real-time monitoring of treatment progress, with a countdown timer and optional notifications is available.

### Connectivity:

The mobile app interfaces with the phototherapy device via WiFi or Bluetooth. The electronic component for WiFi and IR remote application 309 located in the base 300 facilitates this connection.

A secure pairing process ensures that users can connect to their specific devices quickly and securely.

### Custom Treatment Plans:

One of the most compelling features of the mobile application is the ability to create custom treatment plans. Users can save multiple treatment profiles for different skin conditions or family members.

The application also offers preset treatment plans based on scientifically backed research for conditions like acne, wrinkles, and pigmentation.

### App-Remote Synchronization:

All settings configured in the mobile application can be synchronized with the remote control 400, ensuring a seamless experience whether users prefer the tactile feedback of physical buttons or the convenience of a mobile interface.

### Software Updates:

The mobile application allows for over-the-air updates for both the app and the phototherapy device's firmware, ensuring that users always have access to the latest features and performance improvements.

### Security and Data Privacy:

The application is compliant with relevant data privacy laws and utilizes state-of-the-art encryption algorithms to protect user data.

The integration of this mobile application greatly amplifies the functionality, convenience, and personalization capabilities of the multi-wavelength LED phototherapy device 100, making it a comprehensive solution for a broad range of skin conditions.

This mobile application is an integral part of the invention, providing users with unprecedented control and customization over their phototherapy treatments.

By connecting this highly specialized mobile application to the phototherapy device, the invention represents a significant advancement in the field, offering features and functionalities that are designed to improve user experience, treatment efficiency, and overall effectiveness.

### Functionality and Operation

Upon synchronization with the integrated mobile app, the Multi-Wavelength LED phototherapy device with integrated mobile app control 100 can be activated either via the app or the Remote Control 400. Users can select their desired therapeutic light based on their specific skin needs. The device's 888 LED beads 204 in the silicone body 200 then emit the chosen light. This emitted light targets and treats a variety of skin conditions based on the properties of the selected light color. The primary objective of this device is to accelerate blood reflux, enhance skin elasticity, improve skin chlorosis, and dullness, thereby achieving skin whitening, rejuvenation, wrinkle reduction, and spot repairing. This instrument is technologically advanced, utilizing seven distinct cold light sources. Each light type possesses unique attributes and is designed to address particular skin issues.

### Specific Light Functions:

Red Light (630 nm): Boosts cell activity, accelerates metabolism, and promotes collagen formation. Benefits include skin whitening, rejuvenation, wrinkle reduction, skin elasticity enhancement, damaged skin repair, pore reduction, and anti-aging.

Blue Light (470 nm): Offers bactericidal properties, inflammation inhibition, acne and sore removal, sterilization, skin repair, and scar prevention.

Green Light (520 nm): Helps in reducing skin oil secretion, balancing skin, relieving stress, and lymph drainage.

Yellow Light (590 nm): Energizes skin cells, decomposes pigments, promotes lymphatic drainage, improves skin texture, treats skin redness, and enhances immunity.

Purple Light (400-420 nm): A combination of red and blue lights, it's particularly effective in treating acne and scars.

Cyan Light (490-520 nm): Aids in increasing cell energy and boosting metabolism.

Laser Light (white light) (400-700 nm): Penetrates the skin's deeper layers, accelerates skin metabolism, breaks down pigment spots, and addresses fine lines and sagging skin.

By leveraging the distinct properties of each light type, users can customize their treatments based on specific needs and concerns. The device's flexibility ensures that it remains a versatile tool for skin care and therapy.

1. A phototherapy device, comprising:
a silicone body containing an array of LED beads configured to emit multiple distinct wavelengths of light for skin treatment;
a base with electronic components;
a remote control equipped with a time display, a color display, and silicon buttons for device operation;
a mobile application configured to interface with the phototherapy device via WiFi or Bluetooth facilitated by an electronic component in the base, wherein said mobile application allows users to select and customize light wavelength, intensity, and duration for treatment.

2. The phototherapy device of claim 1, wherein the silicone body further includes:
a transparent silicon logo;
a first layer;
a secondary layer of translucent silicon;
an OLED Light situated in a slot for OLED to light the transparent silicon logo;
a 5-pin female port to connect with the 5-Pin connector of the base for power and data transmission.

3. The phototherapy device of claim 1, wherein the array of LED beads installed between the first layer and the secondary layer of translucent silicon.

4. The phototherapy device of claim 1, wherein the base includes:
a cable to 5-Pin Connector for silicon pad;
a cable to IR remote receiver;
an IR remote receiver;
a lighted power button;
LED edge light for functional indicators;
a cable from charging port;
a battery;
an electronic component for WiFi and IR remote application;
a 5-Pin connector;
a charging power port;
an anti slip rubber;
screw location.

5. The phototherapy device of claim 1, wherein the remote control further comprises a light grey printed power symbol.

6. The phototherapy device of claim 1, wherein the mobile application includes features for:
real-time monitoring of treatment progress;
creation and storage of custom treatment plans based on user needs or scientific research;
synchronization of settings with the remote control;
over the air updates for both the mobile application and the device firmware;
security and data protection compliant with relevant laws.

7. The phototherapy device of claim 1, wherein the LED beads are capable of emitting multiple wavelengths spanning the visible light spectrum for therapeutic purposes.

8. A method for using the phototherapy device of claim 1, the method comprising:
activating the device via the integrated mobile app or the remote control;
selecting a desired therapeutic light wavelength based on skin needs using the mobile application or remote control;
administering the selected light treatment to the skin for a pre-determined duration.

9. The method of claim 8 wherein the therapeutic light treatment accelerates blood reflux, enhances skin elasticity, improves skin chlorosis, achieves skin whitening, rejuvenation, wrinkle reduction, and spot repairing.

10. The phototherapy device of claim 1, wherein the silicone body is made of white silicon for aesthetic and skin safety purposes.

11. The phototherapy device of claim 1, wherein the base possesses an aluminum electroplated finish for durability purposes.

12. The phototherapy device of claim 1, wherein the mobile application allows users to pre-program treatment sessions, offering real-time monitoring, countdown timer, and optional notifications.

13. The phototherapy device of claim 1, wherein the mobile application facilitates a secure pairing process for users to connect their specific devices.

14. The phototherapy device of claim 1, designed to reduce the limitations of traditional phototherapy devices by offering the versatility of multiple light wavelengths, user-friendly controls, and enhanced safety for various skin types.

15. The phototherapy device of claim 1, where each type of emitted light from the LED beads addresses specific skin issues or enhances particular skin properties.

16. The phototherapy device of claim 1, wherein the device's primary purpose is to treat conditions including but not limited to acne, wrinkles, pigmentation, and fine lines.
